(19) United States
(12) Patent Application Publication
(10) Pub. No.: US 2024/0285303 A1
(43) Pub. Date: Aug. 29, 2024

(54) SURGICAL CUTTING DEVICE FOR OTOLOGY AND METHOD OF OPERATING THE SAME

(71) Applicant: Heinz Kurz GmbH, Dusslingen (DE)

(72) Inventors: Thomas STIEGELE, Riederich (DE); Alexander SANDER, Titisee-Neustadt (DE); Matthias MERTENS, Kaltenkirchen (DE)

(73) Assignee: Heinz Kurz GmbH, Dusslingen (DE)

(21) Appl. No.: 18/585,135

(22) Filed: Feb. 23, 2024

(30) Foreign Application Priority Data
Feb. 24, 2023 (DE) 10 2023 104 616.0

(51) Int. Cl. A61B 17/3205; A61B 17/00

(52) U.S. Cl. CPC A61B 17/3205; A61B 2017/00787

(57) ABSTRACT

A surgical cutting device in otology for the production of a thin tissue or cartilage slice, has an elongate device body aligned along a longitudinal axis, a handle-like grip, and a cutting head with a cutting blade having a blade body with a sharp cutting edge. The cutting blade is arranged within a cutting window which is continuously open in a direction transverse to the direction of the longitudinal axis. The cutting blade is positioned in the cutting window so that its sharp cutting edge is aligned parallel to a transverse axis which extends perpendicular to the longitudinal axis and obliquely to the direction. The sharp cutting edge is positioned axially away from the blade body in the direction of the longitudinal axis towards the handle-like grip. The cutting blade is connected to the device body either rigidly or movably about the transverse axis.

### CROSS REFERENCE TO RELATED APPLICATIONS

Applicant claims priority under 35 U.S.C. § 119 of German Application No. 10 2023 104 616.0 filed Feb. 24, 2023, the disclosure of which is incorporated by reference.

### BACKGROUND OF THE INVENTION

### 1. Field of the Invention

The invention relates to a medical cutting device in otology for the production of thin slices of tissue or cartilage slices from a patient's own body material for otological applications, with an elongated device body aligned along a longitudinal axis, which device body comprises a handle-like grip at a first end and a cutting head with a cutting blade at the opposite second end, which has a blade body with a sharp cutting edge.

### 2. Description of the Related Art

Such a device is known from Dietrich Plester et al. “Atlas der Ohrchirurgie”, page 79, Verlag Kohlhammer (1989) ISBN 3170084887 (=reference [1]).

Cutting out thin pieces of the body's own tissue, particularly cartilage slices of uniform thickness, from a larger piece of cartilage, e.g. taken from the auricle, the tragus, the cartilaginous part of the upper rib bones or the nasal septum, is an activity that is repeatedly required for various medical purposes. For example, it may be necessary to examine special properties of the base body in more detail, particularly under a microscope. In otorhinolaryngology, however, such thin cartilage slices are also required for many surgical procedures, e.g., in the middle ear region to cover a middle ear prosthesis, to reconstruct the posterior wall of the auditory canal or for the plastic treatment of an eardrum defect. Thin cartilage slices of this kind are also used in many nasal surgical procedures to perform functional or aesthetic corrections to the nose.

Devices for Processing Tissue or Cartilage Slices Made from a Patient's Own Body Material

EP 0 483 567 B1 (=reference [2]) describes a cutting device with which thin cartilage slices of a predeterminable thickness—within certain limits—can be cut out of a larger piece of cartilage quickly, safely and with uniform quality. However, in order to achieve different thicknesses of the cartilage slices produced, special spacer blades of known thickness must be inserted into the cutting device. Just like the cutting device itself, these spacer blades must be kept strictly clean and sterile and treated before each operation, which is both time-consuming and prone to errors. Considering that there are three to four operating theaters in an average ENT clinic and that fifteen to twenty patients are operated on in one day at peak times, a cartilage cutter may have to be provided up to fifteen times in one day. This is a major logistical challenge for sterile supply. In addition, handling the spacer blades is not easy. Due to their small size, for example, it is only possible to label them sufficiently and clearly recognizably to a limited extent, but this is the basic prerequisite for ensuring that exactly the spacer blade of the required size is available during the operation. Correctly inserting and fixing the rather small spacer blades into the cutting device also requires some skill.

In order to be able to produce thin cartilage slices of certain different thicknesses in uniform quality even without using the known spacer blades, it is proposed in US 2010/0286693 A1 (=reference [3]) that—as in the known cutting device according to reference [2]—the first depression arranged in the first working portion on the upper side of the device body can be closed by a first protrusion arranged on the upper side of the cover, wherein the first lateral boundary web has a first guide slot for inserting a cutting blade, which guide slot is extended from a face side of the first portion at a predetermined first distance parallel to the base surface of the first recess. In addition, in the device according to reference [3], at least one second holding apparatus is to be provided, which has a second portion with a second depression arranged on the upper side of the device body, which second depression is completely or partially surrounded by a second boundary web and can be closed by a second protrusion arranged on the upper side of the cover, wherein the second lateral boundary web has a second guide slot for inserting a cutting blade, which guide slot is guided from a face side of the second portion and extends at a predetermined second distance parallel to the base surface of the second recess.

Since the distances between the respective guide slot and the corresponding base surface of the respective recess can be selected differently for different holding apparatuses, this known cutting device can be used to produce cartilage slices of certain different thicknesses with the different holding apparatuses without having to use the spacer blades that were indispensable in the prior art. Furthermore, handling this cutting device between the thumb and index finger gives the surgeon more safety during the actual cutting process because all parts can be moved safely and in a controlled manner in relation to one another due to their geometry and design. However, the disadvantage of this known cutting device is that parts can only be produced with a great deal of effort. Injection-molded products in particular have the property that very thin walls can cause major problems. As a result, the products are generally not economically viable due to the financial outlay involved.

DE 10 2013 105 857 B4 (=reference [4]) improves on this prior art by proposing a modified cutting device, the use of which, however, also poses a certain risk of injury to the person handling it, because the cutting blade is freely accessible before it is inserted into the guide slot and can therefore cut the user's finger in the event of a clumsy movement. A further disadvantage of this known device (as well as the prior art discussed above) is that the blade must be handled with sawing or chopping movements to cut off the desired cartilage slice, which often leads to uneven cutting results.

A cutting device with a rotating cutting blade is known from DE 10 2017 111 634 B3≈EP 3 409 233 B1≈U.S. Pat. No. 10,663,375 B2 (=reference [5]).

A relatively simple, early cutting device in the form of a disposable cartilage cutter is described in EP 2 249 139 B1≈U.S. Pat. No. 8,562,614 B2 (=reference [6]).

An improved embodiment of a disposable cartilage cutter compared to reference [6] is represented in DE 10 2013 105 857 B4≈EP 2 810 628 B1=U.S. Pat. No. 9,532,790 B2 (=reference [7]).

For more durable and repeated use, the applicant shows a precision cartilage cutter on its homepage at https://www.kurzmed.com/de/produkte/otologie/instrumente-und-zubehoer.html whose application is explained for example in https://www.youtube.com/watch?v=NrStxT4JOtU (=reference [8]).

However, all cutting devices in accordance with references [2] to [8] have in common that they only deal with the further processing, particularly the precise cutting of already existing tissue or cartilage parts, but not with the “extraction” of the same from a patient's own body material by means of a surgical procedure.

### Reimplantations in Ophthalmology

Surgical microkeratomes are known for example from DE 698 18 043 T2 (=reference [9]) from the field of ophthalmology, which is relatively remote in terms of the generic otology of the present invention, where not cartilage tissue but very soft or gelatinous corneal or macular tissue of a completely different nature can be removed and reinserted. In this procedure, small parts of the cornea are used to surgically correct eye lens refractive disorders, which can be removed by means of a special cutting tool.

The company Gebauer Medizintechnik GmbH in D-75242 Neuhausen also describes on its website www.gebauermedical.com (=reference [10]) cutting devices for the removal of corneal material by means of a plane-like forward movement in response to pressure from the grip of the device in the direction of a cutting head.

### Surgical Instruments for Scraping and Collecting Bone Particles

US 2007/0208348 A1 (=reference [11]) describes a device for scraping thin bone parts—but not tissue or cartilage—with a cutting blade rigidly connected to the device body. In this device, the cutting blade is arranged within a cutting window which is continuously open in a direction x′ transverse to the direction of the longitudinal axis, wherein the cutting blade is positioned in the cutting window in such a manner that its sharp cutting edge is aligned parallel to a transverse axis which extends perpendicularly to the longitudinal axis and obliquely to the direction x′. The sharp cutting edge of the cutting blade is positioned axially away from the blade body in the direction of the longitudinal axis of the handle-like grip. The bone parts are scraped off by an axial planning movement away from the grip in the direction of the cutting window. Through the opening in the cutting window, the planed bone particles are pushed into a hollow space inside the device where they can be collected.

Prior Art Specific to the Invention from the Field of Otology

In their publication “A Comparative Evaluation of Audiological and Graft Uptake Results of Reinforced Sliced Conchal Cartilage Versus Temporalis Muscle Fascia Graft In Type I Tympoplasty”, International Journal of Clinical & Experimental Otolaryngology (IJCEO), 2018; 4(1): 96-100 (=reference [12]), Singh S P et al. also describe the removal and reimplantation of cartilage tissue in otolaryngology. They show and describe special cartilage cutters for removing the cartilage.

Finally, the reference [1] already mentioned at the beginning describes a surgical cutting device in otology generic to the present invention for the production of a thin tissue or cartilage slice from a patient's own body material for otological applications. In accordance with reference [1], this device consists of a surgical scalpel with all the feature complexes defined at the beginning.

The disadvantage here is that the scalpel must be used freehand when removing tissue or cartilage. The quality of the cutting result is always highly dependent on the surgeon's daily form and skill.

### SUMMARY OF THE INVENTION

The object of the present invention, on the other hand, is to improve a generic otological cutting device of the type described ab by the simplest possible technical means in a simple and cost-effective manner in such a manner that the advantages of the known cutting devices described above are largely retained, but the disadvantages mentioned are avoided. The invention is particularly intended to ensure that the cutting process can be performed in a simple and accident-proof manner, wherein particularly uniform cutting results should be achievable with high-quality cutting products at the end, in particular cartilage or tissue plates with a defined, predeterminable layer thickness.

### BRIEF DESCRIPTION OF THE INVENTION

This relatively complex object is achieved in accordance with the invention, in a surprisingly simple, effective and cost-effective manner, by a surgical cutting device having an elongate device body which is aligned along a longitudinal axis, and which comprises at a first end a handle-like grip and at the opposite second end a cutting head with a cutting blade which has a blade body with a sharp cutting edge. The cutting blade is arranged within a cutting window which is continuously open in a direction x′ transverse to the direction of the longitudinal axis. The cutting blade is positioned in the cutting window in such a manner that its sharp cutting edge is aligned parallel to a transverse axis which extends perpendicularly to the longitudinal axis and obliquely to the direction x. The sharp cutting edge of the cutting blade is positioned axially away from the blade body in the direction of the longitudinal axis z towards the handle-like grip; and the cutting blade is connected to the device body either rigidly or movably about the transverse axis.

This makes it possible to use the otological device according to the invention by pulling, in contrast to the methods used in ophthalmology (references [9], [10]) and the device for bone scraping (reference [11]), where work is carried out by pushing. In such a manner, this sensitive mode of operation is particularly gentle when surgically “processing” delicate living tissue and leads to geometrically much more precise work results.

This design and the resulting mode of operation ensure that the sharp cutting blade always remains concealed within the guide slot of the cutting window during use, so that there is hardly any risk of injury to an operator.

Compared to a simple scalpel, the alignment of the sharp cutting edge parallel to the transverse axis of the device enables a uniform, planar pulling movement of the cutting blade over the location of the removal from the patient. In contrast to the prior art devices described above, this enables considerably improved work results with regard to particularly uniform, reproducible cuts of the tissue or cartilage piece and the extraction of slices or layers with a precisely predetermined thickness. The design of the cutting device according to the invention ultimately leads to significantly better, finer and more even cutting results.

Particular advantages of the device according to the invention over the prior art include:

Simplicity of the device: In the simplest embodiments, the cutting device according to the invention comprises only the grip and the cutting head. Both parts of the device can be manufactured with a high degree of precision, but without any major technical effort.

Precision: The possibility of cutting depth adjustment allows a cut through the patient's tissue that is particularly characterized by uniform cutting thickness and surface quality.

Safety: The blade is not directly accessible to the user, making a cut injury extremely unlikely.

Simple removal of cartilage layer
low physical effort
   A pulling movement results in a lighter pull due to the upward force component (for the use case) than when pushing with the downward force component

Defined layer thickness
An additional work step is not necessary. Until now, a part has been cut out and subsequently adjusted to the layer thickness again with an additional instrument (see references [2] to [8]).

Less removal of cartilage material than with the previous procedure or only to the extent necessary
Gentler procedure for the patient, gentler surgery and better healing process
Only a “chip” is peeled off/harvested, so to speak, without a larger piece of tissue or cartilage having to be cut out.
This procedure is also faster, as the thickness of the peeled (“harvested”) chip does not need to be further processed.

The cutting device according to the invention can be designed as a sterile disposable product.
Elaborate processing is then no longer necessary.

### Preferred Embodiments of the Invention

Advantageously, there is a class of embodiments of the cutting device according to the invention in which the cutting window and the cutting blade are accommodated in a cutting body, which is rigidly, particularly integrally, connected to the device body.

This enables precise dosing and transmission of the force applied when peeling out a piece of tissue or cartilage. This simple design also makes production easier.

Particularly preferred are embodiments of this class of embodiments, in which the cutting body has a spacer which defines the penetration depth of the sharp cutting edge into the patient's own body material and thus the thickness of the thin tissue or cartilage slice that is peeled out.

This makes it possible to “harvest” particularly precise chips of a desired layer thickness, which can usually be used for reimplantation in the patient without further processing.

In order to be able to set the currently required layer thickness particularly precisely and finely in individual cases, in preferred embodiments the spacer is designed to be adjustable, for example with one or more spacer discs, which particularly have a thickness between 0.1 mm and 1 mm, preferably between 0.3 mm and 0.4 mm, which normally satisfies the most common requirements.

Further developments of these embodiments of the invention are also of particular practical advantage, in which the cutting head has a viewing window following the cutting body, which viewing window is axially offset in the direction of the longitudinal axis z towards the handle-like grip and which is continuous in the direction of a vertical axis x perpendicular to the longitudinal axis z and perpendicular to the transverse axis y, as is known per se, for example, from the publication DE 101 38 235 C1 (=reference [13]).

This viewing window allows the surgeon to visually assess and control the removal region on the patient during tissue or cartilage removal.

Particularly preferred are also embodiments of the according to the invention, which are characterized by the fact that a cartilage punching device is arranged at the free end of the handle-like grip opposite the cutting head.

The cartilage punching device is integrated into the cutting device, for example, for the standardized production of a stabilizer for full prostheses. It allows small cartilage slices to be produced quickly intraoperatively and in a single step. In such a manner, transplants are approximately suitable for covering a tympanoplasty prosthesis.

The design of embodiments of the invention in which the cutting blade is arranged in the cutting body with the side of its sharp cutting edge tilted against the z-y plane by a setting angle of between 10° and 40°, preferably approximately 25°, is particularly favorable from an ergonomic point of view.

Particularly cost-effective in production, but also sufficiently reproducible and consistent in quality, are embodiments of the surgical cutting device according to the invention in which the cutting blade is made from a commercially available razor blade.

A further class of advantageous embodiments of the cutting device according to the invention is characterized in that the cutting blade is produced from stainless steel, preferably surgical steel.

This means that the cutting device also meets the highest requirements for clinical hygiene, especially in the area of surgery.

In order to further increase the quality of the cutting device according to the invention, in preferred embodiments of this class of embodiments the sharp cutting edge of the cutting blade has a bevelled polished section on both sides and is preferably specially hardened.

A further class of embodiments of the invention, which are somewhat more complex in terms of material costs, include a cutting device in which the entire device body is made of stainless steel, preferably surgical steel.

However, surgical cutting devices of this kind can be sterilized as often and repeatedly as required in accordance with the highest clinical hygiene standards and can be reused for practically any length of time.

In an alternative class of embodiments of the invention, which are generally somewhat more cost-effective to manufacture and somewhat lighter in weight, parts of the cutting device, particularly the handle-like grip, are made of plastic.

The cutting device according to the invention can also be designed particularly as a low-cost disposable product, which is disposed of after a single use. This eliminates the need to clean and re-sterilize the device. This is becoming increasingly important, as the contamination of surgical instruments is a growing hygiene problem in everyday clinical practice. Therefore, the cutting device according to the invention is particularly preferably produced at least in parts from a sterilizable plastic. This makes the cutting device considerably cheaper to produce than the usual devices made purely of metal. They are then delivered to the surgery in sterile packaging and the used cutting device can simply be disposed of. Such a sterile-packed disposable product also has the advantage that the cutting device does not have to be cleaned and sterilized before each operation and the risk of infection, which cannot be ruled out with a sterile goods supply, is minimized. Preferably, the corresponding parts of the cutting device are produced using an injection molding or 3D printing method.

To further save material and weight, in preferred embodiments of the surgical cutting device according to the invention, the handle-like grip may have a hollow or concave structure.

In practice, embodiments of the surgical cutting device according to the invention in which the sharp cutting edge of the cutting blade has a bar-like or sawtooth-like or sickle-like or oval or round, concave or convex shape, particularly on a circular portion, preferably on a quarter or semicircle, have proven successful.

Also within the scope of the present invention is a method for operating a surgical cutting device of the type described above according to the invention for the production of a thin tissue or cartilage slice, in which the sharp cutting edge of the cutting device is placed on a suitable part of the patient's body with slight pressure, and in that a desired tissue or cartilage slice is peeled out of the patient's own body material with particular sensitivity by pulling the cutting device in the direction of the grip.

The use of the surgical cutting device according to the invention by pulling (instead of pushing) is therefore particularly gentle on the patient and the result is also much more precise chips.

Further features and advantages of the invention are apparent from the following detailed description of embodiments of the invention, with reference to the figures of the drawing, which show details essential to the invention, and from the claims.

The individual features can each be implemented individually or together in any combination in variants of the invention. The embodiments shown and described are not to be understood as an exhaustive list, but, rather, have an exemplary character for the description of the invention.

### BRIEF DESCRIPTION OF THE DRAWINGS

Other objects and features of the invention will become apparent from the following detailed description considered in connection with the accompanying drawings. It is to be understood, however, that the drawings are designed as an illustration only and not as a definition of the limits of the invention.

In the drawings,

FIG. 1A shows a schematic spatial representation obliquely onto the underside of the device body of one embodiment of the cutting device according to the invention;

FIG. 1B shows the same as FIG. 1A, but with an oblique view of the cutting device from above;

FIG. 2A shows the embodiment of FIG. 1B in a view from the side in the direction of the transverse axis y;

FIG. 2B shows the same as FIG. 2A, but viewed from above along the vertical axis x;

FIG. 2C shows the same as FIG. 2B, but viewed from below along the vertical axis x;

FIG. 2D shows the same as FIG. 2A, but viewed from the rear in the opposite direction to the longitudinal axis z;

FIG. 2E shows the same as FIG. 2D, but viewed from the front in the direction of the longitudinal axis z;

FIG. 3A shows the same as FIG. 2A, but in a sectional view along the sectional line A-A looking in the direction of the transverse axis y;

FIG. 3B shows detail B of the embodiment in FIG. 3A;

FIG. 3C shows the same as FIG. 2B, but with the sectional line A-A in the direction of the longitudinal axis z for the sectional view in FIG. 3A;

FIG. 3D shows an enlarged detail C of the embodiment in FIG. 3A;

FIG. 4A shows a schematic special representation of the underside of the device body of an embodiment of the cutting device according to the invention without cartilage punching device in the peeling position with the patient's own cartilage material indicated;

FIG. 4B shows the same as FIG. 3A, but in a sectional view along the sectional line D-D looking in the direction of the transverse axis y in the peeling position with the patient's own cartilage material indicated;

FIG. 4C shows an enlarged detail E of the embodiment of FIG. 4B with an indicated tissue or cartilage slice peeled out of a patient's own cartilage material; and

FIG. 4D shows the same as FIG. 3C, but with the sectional line D-D in the direction of the longitudinal axis z for the sectional view in FIG. 4B.

### DETAILED DESCRIPTION OF PREFERRED EMBODIMENTS

The embodiments of the surgical cutting device 10; 20 according to the invention shown schematically in the figures of the drawing are used for the production of thin tissue or cartilage slices 19′ from a patient's own body material 19. The cutting device 10; 20 is configured for otological applications and comprises an elongate device body 11 aligned along a longitudinal axis z, which at a first end has a handle-like grip 12 and at the opposite second end a cutting head 13 with a cutting blade 14, which has a blade body 14′ with a sharp cutting edge 14″.

The cutting device 10; 20 according to the has the cutting blade 14 arranged within a cutting window 15 which is continuously open in a direction x′ transverse to the direction of the longitudinal axis z. The cutting blade 14 is positioned in the cutting window 15 in such a manner that its sharp cutting edge 14″ is aligned parallel to a transverse axis y, which extends perpendicularly to the longitudinal axis z and obliquely to the direction x′. The cutting blade 14 is connected to the device body 11 either rigidly or movably about the transverse axis y.

The cutting window 15 and the cutting blade 14 are accommodated in a cutting body 13′ that is rigidly connected to the device body 11, particularly in one piece. This cutting body has a spacer 16, which defines the penetration depth of the sharp cutting edge 14″ into the patient's own body material 19 and thus the thickness of the thin tissue or cartilage slice 19′ that is peeled out. The spacer 16 is designed to be adjustable, in the exemplary embodiments shown in the drawing with one or more spacer discs 16′.

Following the cutting body 13′ in the direction of the longitudinal axis z towards the handle-like grip 12, the cutting head 13 has a viewing window 17 that is axially offset in the direction of a vertical axis x perpendicular to the longitudinal axis z and perpendicular to the transverse axis y.

At the free end of the handle-like grip 12 opposite the cutting head 13, a cartilage punching device 18 can also be integrated into the cutting device 10. This punching device is used, for example, to quickly produce a standardized stabilizer for total prostheses from the thin tissue or cartilage slice 19′ peeled out of the patient's own body material 19 using the cutting device 10 according to the invention.

In particularly durable embodiments of the cutting device 10; 20 according to the invention, the entire device body 11 is made of stainless steel, preferably surgical steel.

Alternatively, in other embodiments, which are also suitable as a less expensive and lighter disposable product, parts of the cutting device 10; 20, particularly the handle-like grip 12, may be made of plastic.

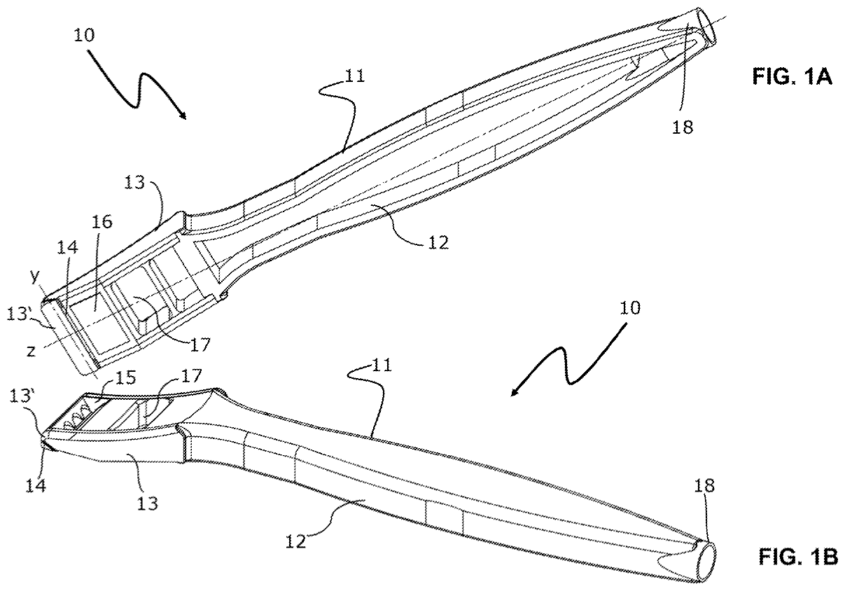

As can be seen particularly clearly in FIGS. 1A, 3A, 4A and 4B, the handle-like grip 12 can also have a hollow or concave structure in order to save weight.

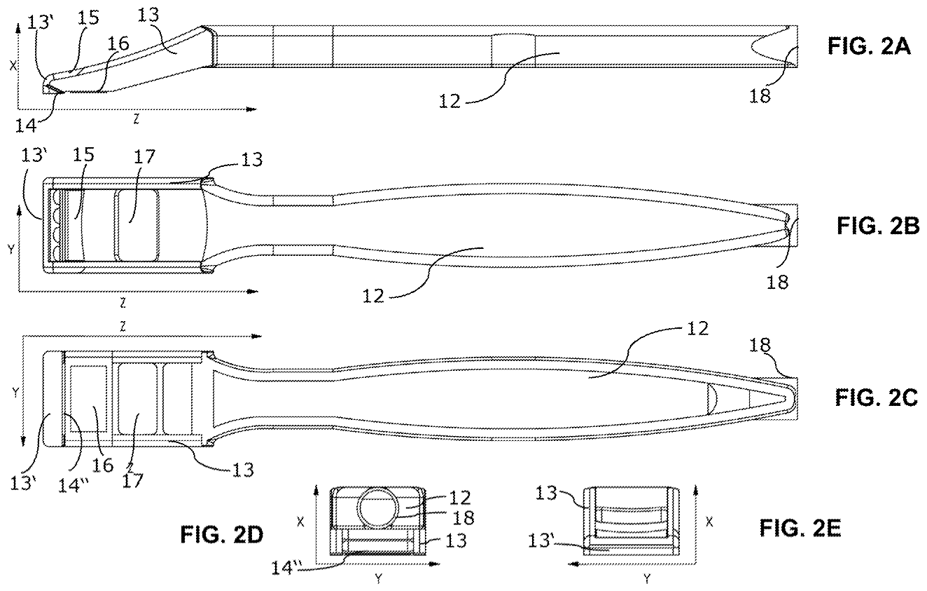

In all embodiments of the cutting device 10; 20 shown in the present drawing, the sharp cutting edge 14″ of the cutting blade 14 has a beam-like structure extending parallel to the transverse axis y, as can be clearly seen particularly from FIGS. 1A and 2C. However, in embodiments of the invention not specifically shown in the drawing, the sharp cutting edge 14″ may also have a different shape, for example a sawtooth-like or a sickle-like or an oval or a round, concave or convex shape, particularly on a circular portion, preferably on a quarter or semicircle.

The cutting blade 14 will generally be made of stainless steel, particularly surgical steel. Particularly in the case of cheaper disposable products, the cutting blade 14 can also be produced from a standard razor blade. Preferably, the sharp cutting edge 14″ of the cutting blade 14 is specially hardened.

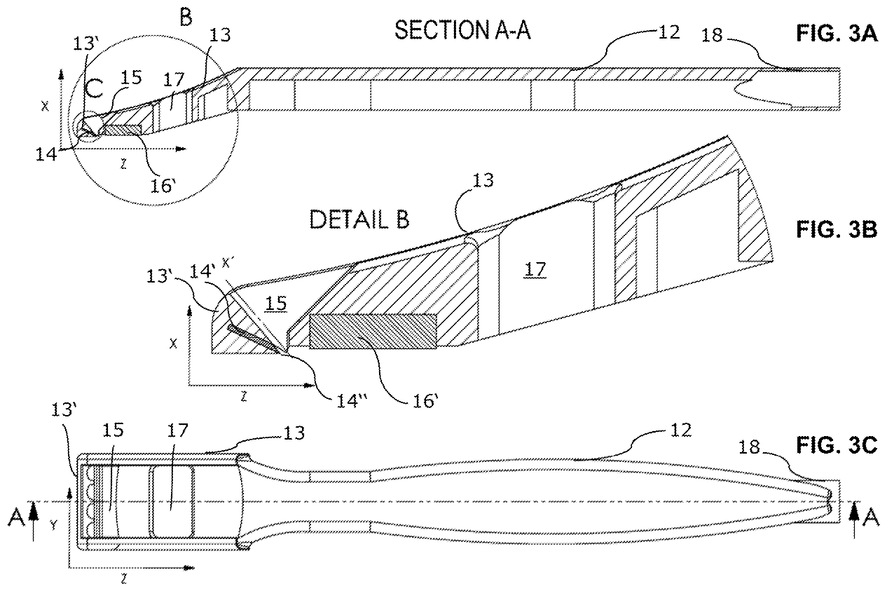

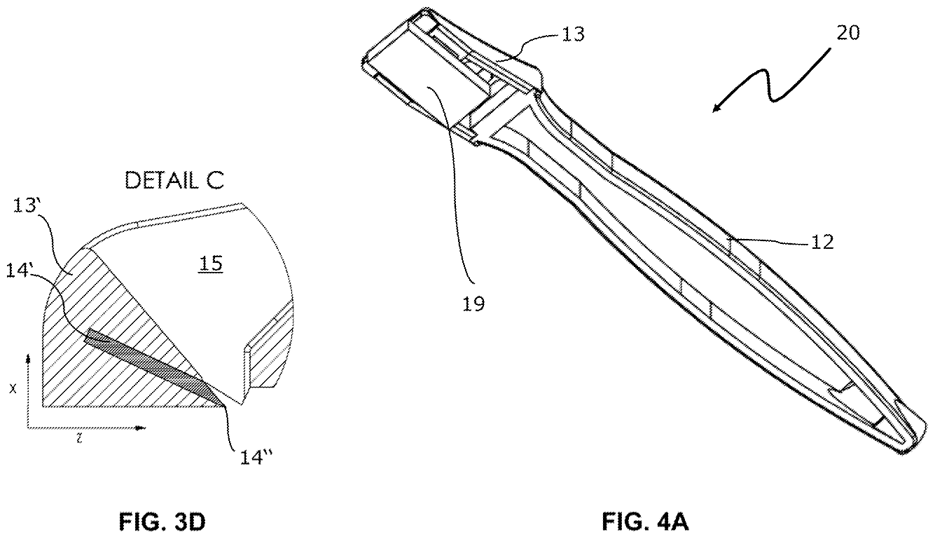

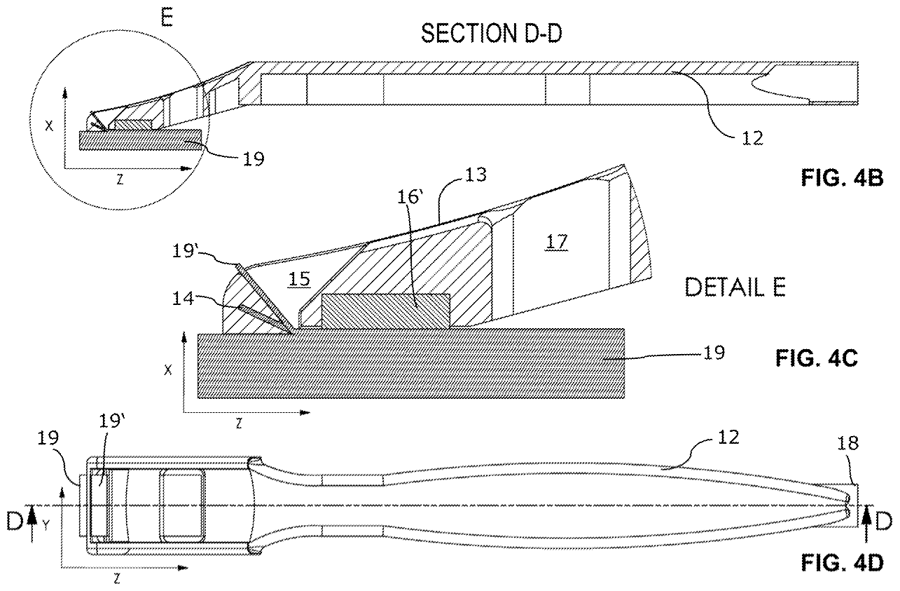

As can be seen particularly clearly in the enlarged detail FIGS. 3B, 3D and 4C, the sharp cutting edge 14″ of the cutting blade 14 can have a single-sided bevel. However, in embodiments of the invention not specifically shown in the drawing, the cutting edge can also have a bevel on both sides.

FIGS. 1B, 2A, 3A, 3B, 4B and 4C clearly show how, in particularly preferred embodiments of the cutting device 10; 20 according to the invention, the cutting blade 14 is arranged in the cutting body 13′ with the side of its sharp cutting edge 14″ tilted at a setting angle of between 10° and 40°, preferably approximately 25°, relative to the z-y plane. In the end region of the cutting head 13 with the cutting body 13′, the inclined course, at least on the underside, then merges again into an end portion extending parallel to the z-y plane, which rests on the corresponding region of the patient's own body material 19 used to peel off a thin tissue or cartilage slice 19′.

FIGS. 2A, 3A, 3B, 3D, 4B and 4C in particular illustrate how the sharp cutting edge 14″ of the cutting blade 14 is positioned axially away from the blade body 14′ in the direction of the longitudinal axis z towards the handle-like grip 12. Thus, a method for operating the surgical cutting device 10; 20 according to the invention for the production of a thin tissue or cartilage slice 19′ can be performed particularly well. Said method is characterized in that the sharp cutting edge 14″ of the cutting device 10; 20 is placed on a suitable part of the patient's body with slight pressure, and that by pulling the cutting device 10; 20 in the direction of the grip 12, a desired tissue or cartilage slice 19′ is peeled out of the patient's own body material 19, as indicated in FIGS. 4A to 4D.

### LIST OF REFERENCE SIGNS

10; 20 surgical cutting device
11 device body
12 handle-like grip
13 cutting head
13 cutting body
14 cutting blade
14′ blade body
14″ sharp cutting edge
15 cutting window
16 spacer
16′ spacer disc
17 viewing window
18 cartilage punching device
19 patient's own body material
19′ thin tissue or cartilage slice peeled out
x′ direction transverse to the longitudinal axis
x vertical axis
y transverse axis
z longitudinal axis

### LIST OF REFERENCES CITED IN THE SPECIFICATION

[1] Dietrich Plester et al. “Atlas der Ohrchirurgie”, page 79 Verlag Kohlhammer (1989) ISBN 3170084887
[2] EP 0 483 567 B1
[3] US 2010/0286693 A1
[4] DE 10 2013 105 857 B4
[5] DE 10 2017 111 634 B3≈EP 3 409 233 B1≈U.S. Pat. No. 10,663,375 B2
[6] EP 2 249 139 B1≈U.S. Pat. No. 8,562,614 B2
[7] DE 10 2013 105 857 B4≈EP 2 810 628 B1≈U.S. Pat. No. 9,532,790 B2
[8] Internet presence of the applicant with precision cartilage cutter https://www.kurzmed.com/de/produkte/otologie/instrumente-und-zubehoer.html
   and
   https://www.youtube.com/watch?v=NrStxT4JOtU
[9] DE 698 18 043 T2
[10] Website of the company Gebauer Medizintechnik GmbH in D-75242 Neuhausen: www.gebauermedical.com
[11] US 2007/0208348 A1
[12] Singh S P et al. “A Comparative Evaluation of Audiological and Graft Uptake Results of Reinforced Sliced Conchal Cartilage Versus Temporalis Muscle Fascia Graft In Type I Tympoplasty”, International Journal of Clinical & Experimental Otolaryngology (IJCEO), 2018; 4(1):96-100
[13] DE 101 38 235 C1

What is claimed is:

1. A surgical cutting device (10; 20) in otology for the production of a thin fabric or cartilage slice (19′) from a patient's own body material (19) for otological applications, comprising:
an elongate device body (11) which is aligned along a longitudinal axis (z), and which comprises at a first end a handle-like grip (12) and at an opposite second end a cutting head (13) with a cutting blade (14) having a blade body (14′) with a sharp cutting edge (14″),
wherein the cutting blade (14) is arranged within a cutting window (15) which is continuously open in a direction (x′) transverse to a direction of the longitudinal axis (z);
wherein the cutting blade (14) is positioned in the cutting window (15) in such a manner that the sharp cutting edge (14″) is aligned parallel to a transverse axis (y), which extends perpendicular to the longitudinal axis (z) and obliquely to the direction (x′);
wherein the sharp cutting edge (14″) of the cutting blade (14) is positioned axially away from the blade body (14′) in a direction of the longitudinal axis (z) towards the handle-like grip (12); and
wherein the cutting blade (14) is connected to the device body (11) either rigidly or movably about the transverse axis (y).

2. The cutting device according to claim 1, wherein the cutting window (15) and the cutting blade (14) are accommodated in a cutting body (13′) which is rigidly, connected to the device body (11), and wherein the cutting body (13′) has a spacer (16) which defines a penetration depth of the sharp cutting edge (14″) into the patient's own body material (19) and thus a thickness of a thin tissue or cartilage slice (19′) which is peeled out.

3. The cutting device according to claim 2, wherein the spacer (16) is designed to be adjustable, for example with one or more spacer discs (16′).

4. The cutting device according to claim 2, wherein the cutting head (13) has a viewing window (17) following the cutting body (13′), which viewing window is axially offset in the direction of the longitudinal axis (z) towards the handle-like grip (12) and which is continuous in the direction of a vertical axis (x) perpendicular to the longitudinal axis (z) and perpendicular to the transverse axis (y).

5. The cutting device according to claim 1, wherein a cartilage punching device (18) is arranged at a free end of the handle-like grip (12) opposite the cutting head (13).

6. The cutting device according to claim 1, wherein the cutting blade (14) is arranged in the cutting body (13′) with a side of the sharp cutting edge (14″) tilted by a setting angle of between 10° and 40° relative to the z-y plane.

7. The cutting device according to claim 1, wherein the cutting blade (14) is produced from a razor blade.

8. The cutting device according to claim 1, wherein the cutting blade (14) is produced from stainless steel.

9. The cutting device according to claim 7, wherein the sharp cutting edge (14″) of the cutting blade (14) has a bevelled polished section on both sides.

10. The cutting device according to claim 1, wherein the entire device body (11) of the cutting device (10; 20) is made of stainless steel.

11. The cutting device according to claim 1, wherein parts of the cutting device (10; 20) are made of plastic.

12. The cutting device according to claim 1, wherein the handle-like grip (12) has a hollow or concave structure.

13. The cutting device according to claim 1, wherein the sharp cutting edge (14″) of the cutting blade (14) has a beam-like or a sawtooth-like or a sickle-like or an oval or a round, concave or convex shape on a circular portion.

14. A method for operating a surgical cutting device (10; 20) for the production of a thin fabric or cartilage slice (19′) according to claim 1, wherein the sharp cutting edge (14″) of the cutting device (10; 20) is placed on a suitable part of the patient's body with slight pressure, and wherein a desired fabric or cartilage slice (19′) is peeled out of the patient's own body material (19) by pulling the cutting device (10; 20) in the direction of the grip (12).

15. The cutting device according to claim 3, wherein the spacer discs have a thickness between 0.1 mm and 1 mm.

16. The cutting device according to claim 15, wherein the spacer discs have a thickness between 0.3 mm and 0.4 mm.

17. The cutting device according to claim 1, wherein the cutting blade (14) is arranged in the cutting body (13′) with a side of the sharp cutting edge (14″) tilted by a setting angle of approximately 25° relative to the z-y plane.

18. The cutting device according to claim 9, wherein the sharp cutting edge is hardened.

19. The cutting device according to claim 11, wherein the handle-like grip is made of plastic.

20. The cutting device according to claim 13, wherein the circular portion is in the form of a quarter circle or semicircle.
